(19) United States
(12) Patent Application Publication
(10) Pub. No.: US 2007/0269499 A1
(43) Pub. Date: Nov. 22, 2007

(54) Materials and methods for wound treatment

(76) Inventors: John Hen, Bradenton, FL (US); John Alfred Thompson, Nassau (BS); Talmadge Kelly Keene, Apollo Beach, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/796,936

(22) Filed: Apr. 30, 2007

Related U.S. Application Data
(60) Provisional application No. 60/796,279, filed on Apr. 28, 2006.

(51) Int. Cl. A61L 15/00; A61K 33/26; A61K 33/38; A61K 33/32

(52) U.S. Cl. 424/445; 424/78.1; 424/647; 424/618; 424/642

(57) ABSTRACT

The subject invention pertains to methods and compositions for wound treatment. The methods and compositions provide for promoting and accelerating wound healing; provide for inhibition of microbial infection; provide for a protective scab-like covering on a wound; and/or for arresting the flow of blood or body fluids from an open wound. The methods and compositions can be used to increase granulation and epithelialization in a wound. In one embodiment, a substantially anhydrous compound of a salt ferrate and a cation exchange material is provided. Compositions of the invention can also include silver compounds. In use, compositions of the invention are preferably applied as a dry dressing to an exuding chronic wound site. If the chronic wound site is dry, the wound site may be wetted with a suitable liquid or aqueous media prior to applying the dressing in dry form.

### CROSS-REFERENCE TO RELATED APPLICATION

This application claims the benefit of U.S. Provisional Application Ser. No. 60/796,279, filed Apr. 28, 2006, which is hereby incorporated by reference in its entirety, including all figures and tables.

### BACKGROUND OF THE INVENTION

In addition to conventional bandages, adhesive means, compresses and the like which are applied with pressure directly against a bleeding open wound, considerable effort has been directed toward the development of chemical agents in various forms that accelerate or enhance the coagulation of blood flowing from an open wound to arrest blood flow. Many of these agents are in the “clotting chain,” i.e., fibrinogen, thrombin, Factor VIII and the like. Others are based upon the use of collagens. Edwardson, in U.S. Pat. Nos. 5,763,411, 5,804,428, and 5,962,026, for example, teaches the use of fibrin in conjunction with a solid support in the and as an enzyme free sealant, and as a solid composition substantially free of catalytic enzymes.

Several patents disclose compositions that promote wound healing in conjunction with a clotting component, including Martin, U.S. Pat. Nos. 5,692,302, 5,874,479, and 5,981,606; Stillwell, U.S. Pat. No. 5,484,913; and Winter et al., U.S. Pat. No. 5,474,782. In U.S. Pat. No. 2,163,588, Cornish teaches a wound pad having very fine fibers carrying a viscous agent and a styptic for arresting and clotting blood flow. Eberl et al., U.S. Pat. No. 2,688,586, teach an improved hemostatic surgical dressing with alginic acid as a clotting agent. Masci et al., U.S. Pat. Nos. 2,772,999 and 2,773,000, also teach hemostatic surgical dressing including a pad and free acid cellulose glycolic acid. A patent for another hemostatic wound dressing is taught by Shelley in U.S. Pat. No. 3,206,361 having an active agent in the form of methylaminoacetocatechol hydrochloride. Likewise, Anderson, in U.S. Pat. No. 3,328,259, discloses a wound dressing containing a film of cellulose glycolic acid ether as the hemostatic agent.

A multitude of other patents, for example Sugitachi et al., U.S. Pat. No. 4,265,233, teach various ready-to-use bandages, pads or other carrying agents containing a hemostatic agents, including Factor VIII, fibrin, thrombin, collagen, polyethylene oxide, epsilon aminocaproic acid (EACA) with calcium chloride, etc. Sakamoto teaches in U.S. Pat. No. 4,655,211 a carrier in the shape of a flake or fiber having thrombin and Factor XIII affixed thereto.

Other patents disclose various fibers capable of inducing clotting. For example, Shimizu et al. in U.S. Pat. No. 5,679,372 teaches an absorbable acetocollagen fibers, while Bell, et al., U.S. Pat. No. 5,800,372, discloses a dressing made of microfibrillar collagen and a superabsorbant polymer for blood absorption and clotting inducement. U.S. Pat. No. 6,521,265 to Patterson and U.S. Pat. No. 6,187,347 to Patterson et al. disclose an admixture of salt ferrate with a cation exchange material that, when hydrated results in the concentration of blood and reduction of Fe⁺⁶ to Fe⁺⁺⁺ to induce clotting.

Chronic wounds present a particularly difficult problem to treat, especially ulcerative wounds such as pressure ulcers (bed sores), diabetic ulcers, venous ulcers, etc. that, without treatment, are often trapped in the inflammation phase of wound healing. These types of wounds often accelerate quickly and damage not only the skin, but underlying tissues as well. They also tend to produce excessive exudates, in addition to blood seepage, during the many weeks or months the wound may take to heal. The excessive healing time required for these types of wounds can lead to secondary complications, such as permanent underlying tissue damage, nerve damage, loss of circulation, and even mortality. In addition, successful treatment of such chronic wounds currently requires more frequent attention by medical professionals, including dressing changes to absorb exudates and control odors and the application of medicaments to control microbial infections. The National Pressure Ulcer Advisory Panel (NPUAP) provides a staging system for clinicians to categorize a pressure ulcer presented by a patient. The four stages are described below.

Stage 1: Pressure ulcer is an observable pressure-related alteration of intact skin whose indicators as compared to an adjacent or opposite area on the body may include changes in one or more of the following: skin temperature (warmth or coolness), tissue consistency (firm or boggy feel), and/or sensation (pain, itching). The ulcer appears as a defined area of persistent redness in lightly pigmented skin, whereas in darker skin tones, the ulcer may appear with persistent red, blue, or purple hues.

Stage 2: Partial thickness skin loss involving epidermis, dermis, or both. The ulcer is superficial and presents clinically as an abrasion, blister, or shallow crater.

Stage 3: Full thickness skin loss involving damage to, or necrosis of, subcutaneous tissue that may extend down to, but not through, underlying fascia. The ulcer presents clinically as a deep crater with or without undermining of adjacent tissue.

Stage 4: Full thickness skin loss with extensive destruction, tissue necrosis, or damage to muscle, bone, or supporting structures (e.g., tendon, joint, capsule). Undermining and sinus tracts also may be associated with Stage IV pressure ulcers.

There remains a need in the art for methods and compositions which provide a protective covering for the wound site, provide prolonged disinfection of a wound site, and/or promote wound healing and/or accelerate coagulation and clotting of blood.

### BRIEF SUMMARY OF THE INVENTION

The subject invention concerns methods and compositions to provide for a protective scab-like covering on a wound, provide for prevention or inhibition of microbial infection in the wound area, accelerate wound healing, and/or to reduce or arrest the flow of blood and other body fluids from an open wound. The subject methods and compositions have been found to be especially useful for the treatment of chronic wounds such as pressure ulcers, venous ulcers, diabetic ulcers, and other exuding wounds, particularly those types of wounds that are unable or slow to heal without treatment. The compositions of the invention increase granulation and epithelialization of tissue in a wound or sore and thereby promote and accelerate healing. In one embodiment, a substantially anhydrous compound is provided comprising a salt ferrate combined with a cation exchange material, and optionally, one or more additional components or agents having antimicrobial, absorptive and/or healing properties. The iron in the salt ferrate has a valency of +6. A composition of the invention is preferably applied as a composition (e.g., as a powder) or dry dressing to an exuding chronic wound site. If the wound site is dry, the wound site may be wetted with sterile water or saline solution prior to applying the composition or dressing in dry form.

In one embodiment, a composition of the invention comprises a cation exchange resin material that is a sulfonated ion exchange resin. In a specific embodiment, the composition includes a substantially anhydrous salt ferrate compound, a cation exchange resin, and a silver containing compound. Compositions of the present invention can be hydrated in the presence of blood, wound exudate, or other selected liquid or aqueous media. Hydration results in the reduction of Fe⁺⁶ to Fe⁺⁺⁺, which promotes clotting of the blood and produces oxygen. Further, the resin produces a scab or protective coating over the wound for protection and further enhances healing. The oxygen produced during the reaction substantially reduces the level of bacteria, virus and fungus at the wound. Additional components in the compound, such as silver Zeolite A, provide long-term anti-microbial action, promote healing and lend additional absorptive properties.

The subject invention also concerns methods for treating an open wound or sore on an animal or human. In one embodiment, the method comprises contacting an exuding open wound or sore with an effective amount of a composition of the invention in a dry state. In another embodiment, the method comprises contacting a dry chronic wound with saline or water or other suitable liquid media first before applying a composition of the invention.

### BRIEF DESCRIPTION OF DRAWINGS

The file of this patent contains at least one drawing executed in color. Copies of this patent with the color drawing will be provided by the Patent and Trademark Office upon request and payment of the necessary fee.

FIG. 1 is a photograph of a patient wound as initially evaluated by a plastic surgeon.

FIG. 2 is a photograph of the wound shown in FIG. 1 after cleansing and surgical debridement and escarotomy. At this stage, the wound has been determined to have a Klebsiella infection.

FIGS. 3A and 3B are photographs of the wound after initial treatment with the composition of the subject invention pursuant to the methods described herein.

FIGS. 4A and 4B are photographs of the wound one week after treatment.

FIG. 5 is a photograph of the wound after cleansing with a saline solution and removal of the overlying scab to evaluate the granulation of tissue.

FIGS. 6A and 6B are photographs of the wound approximately 21 days after continued treatment.

FIGS. 7A and 7B are photographs of the wound approximately 6 weeks after continued treatment.

FIGS. 8A-8F are photographs of a wound over several months of treatment. FIG. 8A shows the wound prior to treatment. FIG. 8B shows the wound on the first day of application of dressing A. FIG. 8C shows the wound 56 days after application of dressing A. FIG. 8D shows the wound 72 days after application of dressing A. FIG. 8E shows the wound 80 days after application of dressing A. FIG. 8F shows the wound 7 months after application of dressing A.

### DETAILED DESCRIPTION OF THE INVENTION

The subject invention concerns methods and compositions to provide for a protective scab-like covering on a wound, provide for prevention or inhibition of microbial infection in a wound area or a medical or surgical procedure site, and/or accelerate healing of a wound or a medical or surgical procedure site by promoting granulation and epithelialization. Compositions of the invention comprise a salt ferrate and an ion exchange resin, such as a cation exchange material, and, optionally, a silver containing compound. Compositions of the present invention increase granulation and epithelialization of a wound. The subject methods and compositions are particularly useful for the treatment of chronic wounds such as pressure ulcers, venous ulcers, diabetic ulcers, and other exuding wounds. A chronic wound typically does not heal in an orderly set of stages and in a predictable amount of time the way most wounds do; wounds that do not heal within three months are often considered chronic. Chronic wounds seem to be detained in one or more of the phases of wound healing. For example, chronic wounds often remain in the inflammatory stage for too long. In acute wounds, there is a precise balance between production and degradation of molecules such as collagen; in chronic wounds this balance is lost and degradation plays too large a role.

The subject invention can also be used to treat dermal lesions, trauma or donor site injuries, incisions, and burns. The compositions of the subject invention are easy to use and to apply to a wound, cause no irritation or allergic responses, and absorb wound exudates (which reduces odors and microbial action at the wound site). If necessary, when the wound is dry (not exuding), the wound site can be wetted first with a suitable liquid, such as sterile water or saline solution before applying the dry composition or dressing. Compositions of the present invention have been shown to accelerate tissue granulation, particularly on stage 2 and stage 3 pressure ulcers, and to increase epithelialization, especially on second and third degree burns. These benefits are further enhanced by the reduction in frequent changes to wound dressings. For most chronic, exuding wounds, use of methods and compositions of the present invention can reduce the frequency of changing a wound dressing from once everyday to once every two to three days or more. As used herein, the term wound refers to a physical trauma wherein skin or muscle or an organ is abraded, lacerated, tom, cut, or punctured, and includes surgical incisions, injection sites, cannulation sites, etc.

The subject invention also concerns methods and compositions for stopping or slowing the flow of blood from an open wound, incision, or medical treatment site. When applied to an open wound for sufficient time, a composition of the invention promotes the clotting of blood and other proteinaceous body fluids and can substantially arrest or minimize further flow of blood and/or body fluid from the wound. In one embodiment, an effective amount of a composition of the invention comprising a salt ferrate, an ion exchange resin, and a silver containing compound is applied to the wound. In a specific embodiment, the salt ferrate is potassium ferrate and the silver compound is silver Zeolite A and the ion exchange resin is a strong acid cation exchange resin, such as a hydrogen form of 2% cross-linked poly(styrene-sulfonic acid) resin.

The subject invention also concerns methods to absorb exudates from an exuding wound, to promote granulation and epithelialization of a wound, and/or to accelerate healing of a wound. Typically, an exuding wound will be a chronic wound. In one embodiment, an effective amount of a composition of the present invention is applied to an exuding wound of a person or animal. In a specific embodiment, the exuding wound is a diabetic ulcer or a decubitis (pressure) ulcer. Optionally, a composition of the invention can be applied dry to a dry (non-exuding) wound that has been wetted first with a suitable liquid or aqueous media, such as a saline solution or sterile water. In a specific embodiment, a composition of the invention is applied to an exuding wound area that has little or no blood present, i.e., the wound does not have uncoagulated blood flowing therefrom. Following application of a composition of the invention, the wound may be left exposed to the air, or the wound may optionally be covered with a bandage or other suitable wound covering.

The dosage or amount of a composition of the invention to be typically administered can be readily determined by an ordinarily skilled clinician and will be dependent on various factors, such as the size and type of wound, the amount of blood or fluid present in the wound, and physical characteristics of the patient, as well as other drugs or treatments the patient is receiving.

The compositions and methods of the present invention can be used to treat any skin sore or wound and are not limited to the treatment of any particular wound, sore, or trauma. The wound or sore to be treated using the present invention can be on the skin or mucus membranes of a patient. The term skin as used herein is understood to include all of the patients' tissue, membranes and the like that can be reached without having to use invasive procedures. Wounds, sores, and lesions that can be treated using the present invention include, but are not limited to, decubitis ulcers, ischaemic leg ulcers, ulcerative keratitis, diabetic ulcers and the like. The present invention can also be used as a therapeutic or prophylactic treatment of bites, stings, and wounds inflicted by animals, such as dog, cat, fish, shark, snake, bat, insect, spider, scorpion, and jellyfish, and plants that contain skin irritants and other toxic chemicals. The present invention can also be used as a therapeutic or prophylactic treatment of burns and surgical incisions, as well as injuries or trauma resulting from exposure to radiation or chemicals.

Methods and compositions of the present invention utilize a substantially anhydrous composition comprising a salt ferrate and a cation exchange material, such as, for example, a sulfonated ion exchange resin. A process for producing ferrates is taught in U.S. Pat. No. 4,545,974, the disclosure of which is incorporated herein by reference. In one embodiment, anhydrous compounds of the present invention comprise a monovalent, divalent, or trivalent salt ferrate (M₂FeO₄, MFeO₄ or M₂(FeO₄)₃) and a cation exchange material. Cation exchangers contemplated within the scope of the invention include water insoluble polymers containing anionic functional groups such as —SO₃⁻, —OPO₃⁻, and —COO⁻. In the practice of this invention, mixtures of insoluble polymers containing different anionic functional groups can be employed. The polymers can be cross-linked. For example, if the polymer is polystyrene, it can be cross-linked with 1% to 10% divinylbenzene. One embodiment of the present invention utilizes an ion exchange resin in the hydrogen ionic form of a sulfonated styrene divinylbenzene copolymer. Methods for preparing ion exchange resins of the invention are disclosed in U.S. Pat. No. 4,291,980, which was based, at least in part, on the production of spherical beads comprised of copolymer styrene and divinylbenzene as taught in U.S. Pat. Nos. 2,366,007 and 3,463,320. The counter ion in the ion-exchange resin can be any cation in the atomic table. The preferred counter-ions include hydrogen, and elements in Groups IA and IIA. While the most preferred cation is hydrogen, mixed cations may be used such as hydrogen and a Group IA element and/or Group IIA element. In another embodiment, the cation-exchange material can be inorganic rather than organically based. Inorganic cation-exchange materials include, but are not limited to, natural or synthetic zeolites, hydrated alkali-aluminum silicates of the general formula M_{2/n}O, Al₂O₃.ySiO₂.wH₂O where M represents a group IA or IIA element, n is the cation valence, y is 2 or greater and w is the number of water molecules contained in the channels within the zeolite. Examples of zeolites are described in U.S. Pat. Nos. 4,911,898; 4,911,899; and 4,938,958. In another embodiment, ion exchange celluloses may be used where the functionalities are classified as strong acid, intermediate acid or weak acid. In another embodiment, mixtures of organic based and inorganic based ion exchangers may be used.

Compositions of the invention are preferably stored under substantially anhydrous conditions and preferably applied as a dry dressing to an exuding chronic wound site. If the chronic wound site is dry, the wound site may be wetted with a suitable liquid or aqueous media, such as sterile water or saline solution prior to applying the composition or dressing in dry form. Compositions of the invention can be provided in a sterile form for application to an open wound.

Examples of elements which can be used as the cation in a salt ferrate of the invention include: H (hydrogen), Li (lithium), Na (sodium), K (potassium), Rb (Rubidium), Cs (Cesium), and Fr (Francium). In one embodiment, the salt ferrate used in a compound of the invention is potassium ferrate (K₂FeO₄). It is known in the art (see U.S. Pat. No. 4,545,974) that the decomposition by hydration of potassium ferrate produces the finest particles of iron oxide (Fe₂O₃) available through the following chemical reaction.
2K₂FeO₄+2H₂O→4 K⁺OH⁻+Fe₂O₃+³/₂O₂↑ (I)

Compositions of the invention can comprise one or more different salt ferrates and in different amounts. For example, in one embodiment, a composition of the invention may comprise potassium ferrate and sodium ferrate in equal or different amounts.

Other cations and cationic groups that can be utilized in a salt ferrate of the present invention include:

TABLE I

| | | | | | |
|---|---|---|---|---|---|
| Be | Beryllium | Mg | Magnesium | Ca | Calcium |
| Sr | Strontium | Ba | Barium | Ra | Radium |
| Ti | Titanium | V | Vanadium | Cr | Chromium |
| Mn | Manganese | Fe | Iron | Co | Cobalt |
| Ni | Nickel | Cu | Copper | Zn | Zinc |
| Ga | Gallium | Ge | Geranium | Zr | Zirconium |
| Nb | Niobium | Mo | Molybdenum | Tc | Technetium |
| Ru | Ruthenium | Rh | Rhodium | Pd | Palladium |
| Ag | Silver | Cd | Cadmium | In | Indium |
| Sn | Tin | Hf | Hafnium | Ta | Tantalum |
| W | Tungsten | Re | Rhenium | Os | Osmium |
| Ir | Iridium | Pt | Platinum | Au | Gold |
| Hg | Mercury | Tl | Thallium | Pb | Lead |
| Bi | Bismuth | Al | Aluminum | As | Arsenic |
| NH₄ | Cation | N(C₄H₉)₄ | Cation | | |

In those embodiments utilizing the K₂FeO₄ as the salt ferrate, or when the cation of the ferrate is H, Li, Na, Rb, Cs, or Fr, it can be understood from Equation I that hydroxide (OH)⁻ radicals are produced. The hydroxide (OH)⁻ radicals remain present in Equation I. It is the presence of the hydroxide (OH)⁻ radicals that can cause a stinging, burning, or otherwise painful sensation when the composition is applied to a wound site. However, use of a salt ferrate having one of the cations listed in Table I produce a slightly altered chemical reaction which neutralizes all of the hydroxide ions produced. For example, using a calcium cation to replace the potassium cation in the salt ferrate, the following chemical reaction occurs:
2CaFeO₄+2H₂O→2Ca(OH)₂+Fe₂O₃+³/₂O₂↑ (II)
As can be observed from Equation II, the produced hydroxide ions are neutralized and combined with calcium. Very little free hydroxide anion is available because of the limited solubility of calcium hydroxide in water. Thus, in one embodiment, a salt ferrate compound of the subject invention utilizes one or more of the cations described in Table I.

Compositions of the subject invention can also comprise additional optional compounds or agents that provide for increased anti-microbial, absorptive, and/or wound healing properties. In one embodiment, a composition of the invention comprises a salt ferrate, a cationic exchange resin, and a silver compound. Silver compounds include, but are not limited to, silver metal (such as nano-silver); silver chlorides; silver oxides; silver sodium hydrogen zirconium phosphate; and silver/zinc form of Zeolite A. In one embodiment, silver/zinc Zeolite A is encapsulated with a random cross-linked polymer of vinyl pyrrolidine and methyl methacrylate. In another embodiment, a composition comprises a silver ion form of cation exchange resins such as cross-linked poly(styrene-sulfonic acid) ion-exchange resin. A specific embodiment of a composition of the invention comprises an admixture of a substantially anhydrous salt ferrate compound, a sulfonated ion exchange resin, and a coated sodium aluminosilicate (Zeolite A) with silver, zinc and ammonium (silver Zeolite A), where the coating consists of a cross-linked polymer of vinyl pyrrolidone and methyl methacrylate. In a specific embodiment, a composition of the invention comprises potassium ferrate combined with the hydrogen form of a 2% cross-linked polystyrene-sulfonic acid resin and silver Zeolite A in the following ratios:

% by Weight

| | | Preferred |
|---|---|---|
| | Approximate | Approximate |
| Component | Ranges | Ranges |
| Potassium Ferrate (KF) | 2-30 | 5-15 |
| H⁺ polystyrene-sulfonic acid resin | 40-97 | 75-92.5 |
| Coated silver Zeolite A | 1.0-30 | 2.5-10 |

Additional components of compositions of the present invention can include, for example, one or more of: zinc compounds, manganese compounds, calcium compounds, and/or copper compounds or derivatives thereof. Examples include, but are not limited to, zinc oxide, zinc sulfate, zinc stearate, manganese oxide, manganese sulfate, manganese citrate, calcium oxide, calcium sulfate, calcium citrate, calcium carbonate, cuprous sulfate; alginates, carrageenans, and agars; chitosan; absorption polymers such as cross-linked polyacylates and acylate copolymers; natural and/or synthetic gums, such as guar, arabic, or karaya; oxidized celluloses; starches, such as tapioca; and drugs, such as antibiotics.

Although the methods and compositions of the present invention greatly enhance blood clotting, inhibit microbial infection, and accelerate wound healing, the wound nonetheless remains open and generally unprotected unless the composition is combined with a covering or carrier such as a BAND-AID, bandage, cotton gauze and the like. In one embodiment, a wound treated with a composition of the invention is subsequently covered with a suitable wound covering or dressing. In another embodiment, a wound covering or dressing is impregnated or coated with a dry powder form of a composition of the present invention and is applied to the wound. Thus, the present invention can also be practiced in conjunction with wound coverings, dressings, and protective materials, such as BAND-AIDS, bandages, cotton gauze, and the like.

The subject invention also concerns kits comprising in one or more containers or packages a composition of the present invention. In one embodiment, a composition of the invention is packaged in a container that is designed in a manner so as to preserve the anhydrous nature of the composition until the container is opened. A kit of the invention can also comprise a container having a quantity of suitable liquid or aqueous media for application to a dry or nonexuding wound prior to application of a composition or dressing of the invention to the wound site. Preferably, the liquid or aqueous media is provided in sterile form. A kit of the present invention can also comprise a wound covering, dressing, or other wound or surgical site protective material, preferably maintained in sterile form until the package or container is opened for use.

The subject invention also concerns wound and surgical site treatment coverings, dressings, and the like. In one embodiment, a dressing of the invention comprises a pad that contains a composition of the invention within and/or on the surface of the pad. In a specific embodiment, the pad is composed of porous foam that is sufficiently open to allow a free flow of powder to fill the voids in the porous foam. The open voids can either be random (like a foam air conditioning filter) or organized into tunnels. The tunnels can keep compositions from mixing until needed. The tunnels can be round holes or geometric shapes. Around the perimeter of the randomly open foam a less porous border may be used to contain the composition. The pad can be designed so that lateral pressure can compress the foam or tunnels and hold the composition in place for inverted application.

In another embodiment, a dressing of the invention comprises a pad with fibers perpendicularly oriented to the plane of the pad, wherein the fibers can hold and release a composition of the present invention. The dressing can be provided with or without an integrated foam or fabric or substrate backing. The dressing can be pre-loaded with a composition of the present invention. The dressing can be of a design wherein the fibers remain attached to the dressing during and/or after application to a wound or surgical site.

In one embodiment, a wound dressing of the invention comprises a flocked pad wherein the pad has a foam (e.g., polyurethane) portion and a flocked fibers portion. In one embodiment, the foam portion is a porous foam as described above. In this embodiment, a composition of the invention can be loaded onto the side of the foam opposite that of the fibers and the composition could then travel or flow through the foam and onto the fibers. The fibers can be attached to the foam portion and can be made, for example, out of calcium alginate. The fibers can be a woven or non-woven material. The fibers can be composed of any suitable material such as cotton, wool, etc. In one embodiment, the fibers are composed of a velvet fabric. The fibers can be coated or flocked with a composition of the present invention. Optionally, the fibers can be composed of dissolvable material (e.g., polyvinyl alcohol) or a biodegradable material (e.g., starch, calcium alginate, polysaccharides, etc.). In one embodiment, the fibers can be composed of a material that can dissolve in a solution, such as a saline solution. In another embodiment, the fibers themselves do not dissolve in solution but are attached to the pad portion via a substance or material that itself can dissolve in solution. This permits a solution to be contacted with a dressing of the invention that has been applied to a site where blood has coagulated and formed a scab, wherein the fibers dissolve or the attachment dissolves and the pad portion of the dressing can then be easily removed without ripping the scab off the wound.

In another embodiment, a dressing of the invention comprises an “island” dressing wherein the dressing has a hollow or open center area that is positioned over the wound or medical treatment site and wherein a composition of the invention can be applied once the dressing is applied to the wound or treatment site. Alternatively, the dressing can have a composition of the invention pre-loaded into the center of the dressing prior to application to a wound or treatment site, wherein the composition is held in place in the dressing by a suitable material that can be removed prior to use of the dressing or that can dissolve in solution or upon contact with blood at the wound or treatment site. The island dressing can be of any suitable size, and shape, and thickness appropriate for the wound site or medical procedure being performed. Preferably, the hollow center portion of the dressing where the composition of the invention is to be applied is larger in diameter and/or circumference than the wound site or procedural site being treated. The dressing can be circular, oval, square, rectangular, diamond, trapezoid, triangular, or any other shape, including irregular shapes. The dressing can be composed of any suitable material including, but not limited to, foam, cork, plastic, woven fiber, compressed cotton, and paper materials.

In another embodiment, a dressing of the invention comprises a pouch or other container that contains a composition of the invention and wherein at least one surface of the pouch or container that contacts the wound or treatment site is dissolvable or biodegradable in blood, bodily fluids, exudates, or other liquids or solvents. In one embodiment, a pouch can be composed of paper or paper blends, polypropylene, or polyvinyl alcohol. In a further embodiment, a composition of the invention is provided in a paste formulation. Carriers that can be used in a paste of the invention include long chain hydrocarbons that impart body, such as, for example, mineral oil and petroleum jelly.

In using a composition of the invention that comprises a salt ferrate and an ion exchange resin, an additional benefit of scabbing or depositing of a substance produced by the reaction with blood or liquid or aqueous media is accomplished over the open wound. This protective coating assists in the production of scabbing over a wound which protects the underlying tissues and promotes healing. In one embodiment, styrene divinylbenzene copolymer is utilized as the ion exchange resin. Details of the composition and method of producing styrene divinylbenzene resin are disclosed in the previously referenced patents and are herein incorporated by reference. In addition to enhancing scabbing of a wound and providing a protective coating, the presence of small amounts of the hydrogen ionic form of styrene divinylbenzene copolymer resin is sufficient to neutralize the hydroxide (OH)⁻ radicals that may be created by hydration of certain subject compositions of the invention. As indicated herein, neutralizing these hydroxide ions reduces or eliminates the stinging effect that may accompany use of a composition of the invention.

In addition, the ion exchange resin of a composition of the present invention plays an active role in the healing of a chronic, exuding wound. A chronic and exuding wound such as a pressure ulcer does not heal by itself. Even with treatments, recovery time depends on the severity of the wound (e.g., stage 2 to stage 3) and can take many months. Stage 4 pressure ulcers can lead to mortality. The compositions of the present invention absorb the exudates and form a loose, somewhat moist coating over the ulcer bed. Pressure ulcers at or above stage 2 in severity continue to release exudates over weeks or months before total healing. The dressing is typically changed (or more dressing is added) on a regular schedule (either once a day to once in 2 to 3 days). Compositions of the present invention actively participate in the healing of chronic, exuding wounds and help absorb any exudate. A chronic wound (e.g., a pressure ulcer) is one which is trapped in the inflammation phase of healing. If the wound is chronic and no treatment is applied, the wound cannot move from the inflammation phase to the granulation phase (the next phase of healing) and the wound worsens. The role of compositions of the present invention is viewed as allowing the transition from the inflammation phase to the granulation phase to occur. Case studies have shown that both the granulation phase and the epithialization phase occur with the use of the present invention to treat stage 2 and stage 3 pressure ulcers. This indicates that the present invention accelerates healing in a chronic wound in comparison to the time required for healing of an untreated wound.

The methods and compositions of the present invention can be used in the treatment of humans and other animals. The other animals contemplated within the scope of the invention include domesticated, agricultural, or zoo- or circus-maintained animals. Domesticated animals include, for example, dogs, cats, rabbits, ferrets, guinea pigs, hamsters, pigs, monkeys or other primates, and gerbils. Agricultural animals include, for example, horses, mules, donkeys, burros, cattle, cows, pigs, sheep, and alligators. Zoo- or circus-maintained animals include, for example, lions, tigers, bears, camels, giraffes, hippopotamuses, and rhinoceroses.

The dosage or amount of a composition of the invention to be typically administered or applied to a site can be readily determined by an ordinarily skilled clinician and will be dependent on various factors, such as the size and type of wound or the surgical or medical procedure being performed, the amount of blood or fluid present in the wound or treatment site, and physical characteristics of the patient, as well as other drugs or treatments the patient is receiving.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### EXAMPLE 1

### Case Study

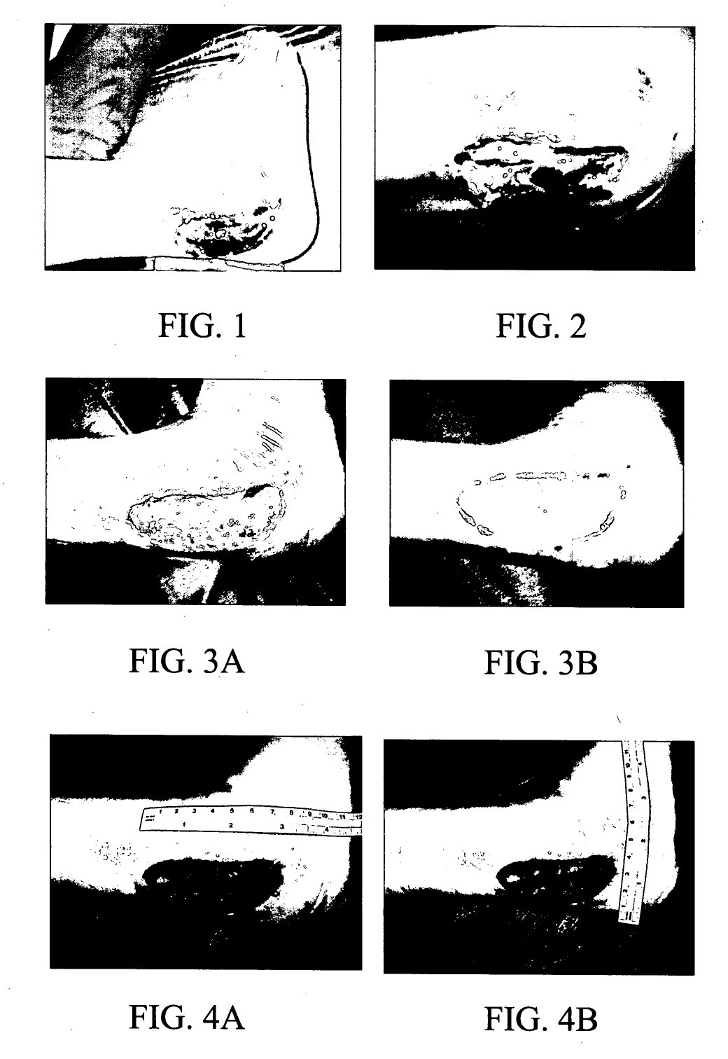

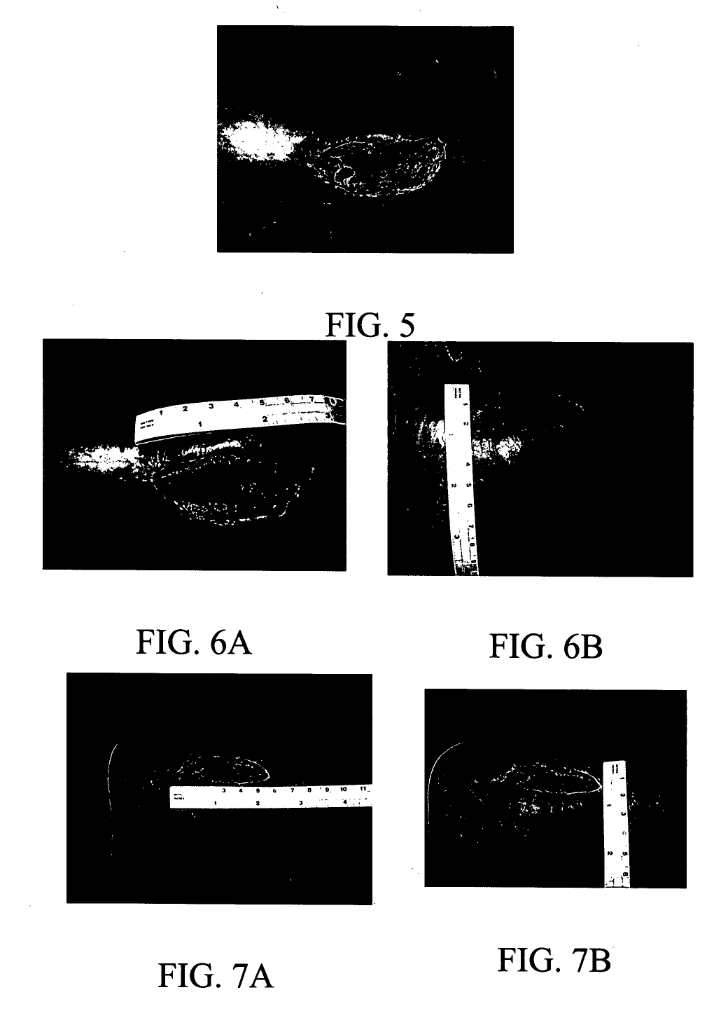

A study with a composition of the invention comprising one part potassium ferrate and seven parts of hydrogen resin (2% cross-linked poly(styrene-sulfonic acid) was conducted on a 36 year old female patient who had had a bursectomy on the right inferior malleolar. She suffered a subsequent dehiscience of cutaneous sutures (rupture of sutures) that developed into a malleolar ulcer. Her orthopedic surgeon referred her to plastic surgeons for treatment of the ulcer. The patient was examined on Nov. 21, 2005. The malleolar ulcer was found to be in Stage 2 covering an area of 8 cm by 3 cm. On Nov. 30, 2005, the wound was cleansed and surgical debridement and escarotomy were performed. The wound was found to be infected with Klebsiella. Treatment with the test composition was started on Dec. 5, 2005 by spreading the powder over the wound site. After one week, the wound was measured, cleansed with saline solution; and the scab was removed to evaluate the progress of granulation tissue. The dimensions of the wound were reduced to 7.5 cm by 2.8 cm. The normal application of the test composition was done once every 2 to 3 days. Evaluation of granulation tissue can only be observed when the coating/dressing is removed. The test composition was applied for another 3 weeks. On Dec. 26, 2005 the wound dimensions was reduced to 6.5 cm by 2.4 cm. After another 3 weeks on Jan. 16, 2006 the wound dimensions was significantly reduced to 5.2 cm by 1.6 cm. Clear evidence of granulation and epithelialization was observed indicating accelerated healing. The healing was accelerated by 2 to 4 weeks when compared to an anti-bacterial Fucidin cream, a standard of care for similar ulcers. FIGS. 1-7 are photographs chronicling the progress of the treatment with the test composition. Table II below chronicles the treatment regime.

TABLE II
Treatment Regime

| | | Dimensions of | % size |
|---|---|---|---|
| Date | Event | ulcer | reduction* |
| Nov. 30, 2005 | Wound cleansed, | 8 cm × 3 cm | 0 |
| | debrided and eschar | | |
| | removed | | |
| Dec. 05, 2005 | Test composition first | 8 cm × 3 cm | 0 |
| | applied | | |
| Dec. 12, 2005 | Cleansed, scab removed, | 7.5 cm × 2.8 cm | 12.5 |
| | test composition | | |
| | continued | | |
| Dec. 26, 2005 | Treatment with test | 6.5 cm × 2.4 cm | 35 |
| | composition continued | | |
| Jan. 16, 2006 | Treatment with test | 5.2 cm × 1.6 cm | 65 |
| | composition continued | | |

*wound dimension approximated as a rectangle

### EXAMPLE 2

### Preliminary Report on a Case of Pressure Ulcer

An elderly patient at the Residencia Medica el Olivar (RMEO), a multipurpose geriatric medical unit in Lima, Peru, was treated with a composition comprising potassium ferrate and a hydrogen form of 2% cross-linked poly(styrene-sulfonic acid) resin (hereinafter the subject composition). Under this protocol patients must have two or more ulcers. One of the ulcers receives the subject composition while the other ulcers serve as controls, receiving the standard medical and surgical care.

The case (LB1) was of a centenarian caucasic female patient that developed multiple wounds in the shin and calf of the left leg. The leg was only mildly swollen but the ulcers had an indurated border that was red, elevated and mildly tender. There was some yellowish secretion at its borders. The ulcer was stage 2a, (minimal depth), which allowed taking two dimensional measurements and photos during a month of comprehensive nursing care. Peripheral pulses were present. Homman sign (for calf vein clots) was negative and there was acceptable venous return. It was thought that leg crossing or another trauma could be playing a role in the development of this wound, however localized vasculitis or a combination of age related micro-vascular events could be playing a role too. One of the anterior wounds was allocated to receive treatment with the subject composition. Results of healing rates can be seen in Table III. The patient's general medical status improved during the procedure. The wound treated with the subject composition granulated faster and “filled in” the gap (a thin epithelium growth ensued) in contrast to the control wound that did not heal at all.

TABLE III
ULCER SIZE (cm)

| | Length (Subject | Width (Subject | Length | Width |
|---|---|---|---|---|
| WEEK | Composition) | Composition) | (Control) | (Control) |
| 1 | 2.5 | 2.5 | 2.0 | 1.5 |
| 2 | 1.5 | 1.5 | 2.0 | 1.5 |
| 3 | 1.0 | 1.0 | 2.0 | 1.5 |
| 4 | 1.0 | 1.0 | 2.0 | 1.5 |
| 5 | 0.5 | 0.5 | 2.0 | 1.5 |

### EXAMPLE 3

### Antimicrobial Activity of Compositions

Compositions of the subject invention have antimicrobial properties. In an exemplified embodiment, a composition comprising seven parts of the hydrogen form of a 2% cross-linked polystyrene-sulfonic acid resin and one part of potassium ferrate was submitted to STS-Duotek, an independent FDA approved laboratory, to test its in vitro activity against the following five microbes.
1. Staphylococcus aureus, ATCC No. 6538
2. Pseudomonas aeruginosa, ATCC No. 9027
3. Escherichia coli, ATCC No. 8739
4. Candida albicans, ATCC No. 10231
5. Methicillin Resistant Staphylococcus aureus (MRSA), ATCC No. 33591

Testing was based on U.S. Pharmacopeia Test Number 51—Antimicrobial Effectiveness Testing; pages 1809 to 1811, USP 24 NF19 U.S. Pharmacopeia & National Formulary—Year 2000, Published by U.S. Pharmacopeial Convention Inc. The results provided in Table IV indicate that the test composition effectively killed the five microbes. All (four) bacterial species and Candida albicans, a yeast, exhibited a 5.5 log reduction or greater within one hour of exposure to the test compound. The log reduction for the bacteria and yeast represented the lower limit of detection in the test design as the initial challenge of each species was 10⁵ cfu.

TABLE IV
Antimicrobial Effectiveness Testing

| | | |
|---|---|---|
| Challenge Organism | Exposure Time | Log Reductions |

Bacteria

| | | |
|---|---|---|
| S. aureus ATCC 6538 | 1 hour | 5.5 |
| | 24 hours | 5.5 |
| | 7 days | 5.5 |
| MRSA ATCC 33591 | 1 hour | 5.5 |
| | 24 hours | 5.5 |
| | 7 days | 5.5 |
| E. Coli ATCC 8739 | 1 hour | 5.5 |
| | 24 hours | 5.5 |
| | 7 days | 5.5 |
| P. aeruginosa ATCC 9027 | 1 hour | 5.6 |
| | 24 hours | 5.6 |
| | 7 days | 5.6 |

Fungi

| | | |
|---|---|---|
| C. albicans ATCC 10231 | 1 hour | 5.7 |
| | 24 hours | 5.7 |
| | 7 days | 5.7 |

### EXAMPLE 4

### Antimicrobial Activity of Compositions with Silver

A composition comprising seven parts of the hydrogen form of a 2% cross-linked polystyrene-sulfonic acid resin, one part of potassium ferrate and variable amounts of silver oxide and silver metal (see Table V) tested for in vitro activity against Staphylococcus aureus (ATCC No. 6538).

24 hours after the compositions were first challenged with the microbe, the test suspensions were re-challenged with microbe and the activity followed for 2 hr, 24 hr and 7 days.

Testing was based on U.S. Pharmacopeia Test Number 51—Antimicrobial Effectiveness Testing; pages 1809 to 1811, USP 24 NF19 U.S. Pharmacopeia & National Formulary—Year 2000, Published by U.S. Pharmacopeial Convention Inc. The results provided in Table V indicate that the test compositions effectively killed the Staphylococcus aureus even after re-challenge. All the test compositions exhibited a 5.5 log reduction within two hours of exposure to the test compound and a 4.6 log reduction after re-challenge. The log reduction for the bacteria represented the lower limit of detection in the test design as the initial challenge of each species was 10⁵ cfu.

TABLE V
Antimicrobial Effectiveness Testing Against S. aureus,
ATCC No. 6538

| 7 parts Hydrogen Resin + | | Log |
|---|---|---|
| 1 part K Ferrate | Exposure Time (hours) | Reductions |
| With 0.05% silver oxide | 2 hrs | 5.5 |
| | 24 hrs | 5.5 |
| | 2 hrs after rechallenge | 4.6 |
| | 24 hrs after rechallenge | 4.6 |
| | 7 days after rechallenge | 4.6 |
| With 0.10% silver oxide | 2 hrs | 5.5 |
| | 24 hrs | 5.5 |
| | 2 hrs after rechallenge | 4.6 |
| | 24 hrs after rechallenge | 4.6 |
| | 7 days after rechallenge | 4.6 |
| With 0.20% silver oxide | 2 hrs | 5.5 |
| | 24 hrs | 5.5 |
| | 2 hrs after rechallenge | 4.6 |
| | 24 hrs after rechallenge | 4.6 |
| | 7 days after rechallenge | 4.6 |
| With 0.20% silver metal | 2 hrs | 5.5 |
| (0.1 um) | 24 hrs | 5.5 |
| | 2 hrs after rechallenge | 4.6 |
| | 24 hrs after rechallenge | 4.6 |
| | 7 days after rechallenge | 4.6 |
| 30% Hydrogen Resin Replaced | 2 hrs | 5.5 |
| with Ag Resin | 24 hrs | 5.5 |
| | 2 hrs after rechallenge | 4.6 |
| | 24 hrs after rechallenge | 4.6 |
| | 7 days after rechallenge | 4.6 |

### EXAMPLE 5

### Antimicrobial Activity of Composition with Alphasan RC2000

Compositions were prepared which were comprised of seven parts of the hydrogen form of a 2% cross-linked polystyrene-sulfonic acid resin, one part of potassium ferrate and variable amounts (2.5%, 5%, and 10%) of ALPHASAN RC2000, a zirconium phosphate-based ceramic ion-exchange resin containing silver. ALPHASAN was obtained from Milliken Chemical (Spartanburg, S.C.) and is an antimicrobial additive that comprises about 10% silver ion by weight. The wound dressing compositions were tested for antimicrobial activity against Staphylococcus aureus ATCC No. 43300 using the Zone of Inhibition Assay (ZOI).

Repeat ZOI Assay

An overnight culture of the test microbe was diluted into saline to a concentration of ca. 1×10⁶ cells/ml. Petri dishes containing Diagnostic Sensitivity Test (DST) Agar were inoculated with 0.2 ml of the cell suspension and incubated for 1 hour. Samples were tested against Staphylococcus aureus (ATCC No. 43300). The powder dressing was poured into the center of the agar plate forming a 1.5 cm diameter circle. The agar plate was incubated for 24 hours at 37° C. Each day, the average inhibition zone (distance between edge of sample and microbial growth) was calculated. After measurements were taken, the sample was transferred to a freshly inoculated agar plate and the assay was repeated.

The results of the ZOI assay showed none of the microorganisms survived exposure for the three wound dressing compositions (containing 2.5%, 5%, and 10% of ALPHASAN RC2000).

### EXAMPLE 6

### Healing of Wounds in Swine

Wound dressing “A” was prepared with 84% hydrogen form of a 2% cross-linked polystyrene-sulfonic acid resin, 12% potassium ferrate and 4% of AJ10D E50 Antimicrobial Powder from AgION Technologies (Wakefield, Mass.). AJ10D E50 is sodium aluminosilicate, silver, zinc, and ammonium encapsulated in a cross-linked polymer of vinyl pyrrolidone and methyl methacrylate. AJ10D E50 has approximately 2.5% by weight silver ion and 14% by weight of zinc ion. Wound dressing “B” compositions comprised 95% cross-linked polystyrene-sulfonic acid resin, 3% potassium ferrate and 2% AJ10D E50. Wound dressings “A” and “B” were used as treatments for a full thickness wound using an impaired wound healing model in a female swine.

Swine Protocol.

The swine was dosed with a steroid. After three days, a series of full thickness wounds were created. The wounds were allowed to develop and mature for 24 hours. The wounds were cleansed and 0.5 grams of each dressing was applied on separate sites, and the treated wounds were wrapped in occlusive bandage to keep materials in contact with the wound. A schedule of cleansing and reapplication of the dressings was followed. After ten days, the wound receiving Treatment A (treatment with wound dressing A) resurfaced (epithelial resurfacing) faster than the wound receiving Treatment B (treatment with wound dressing B). Per cent healed for Treatments A and B was 31% and 20%, respectively, after 10 days.

### EXAMPLE 7

### Healing of Venous Ulcers with Wound Dressing A

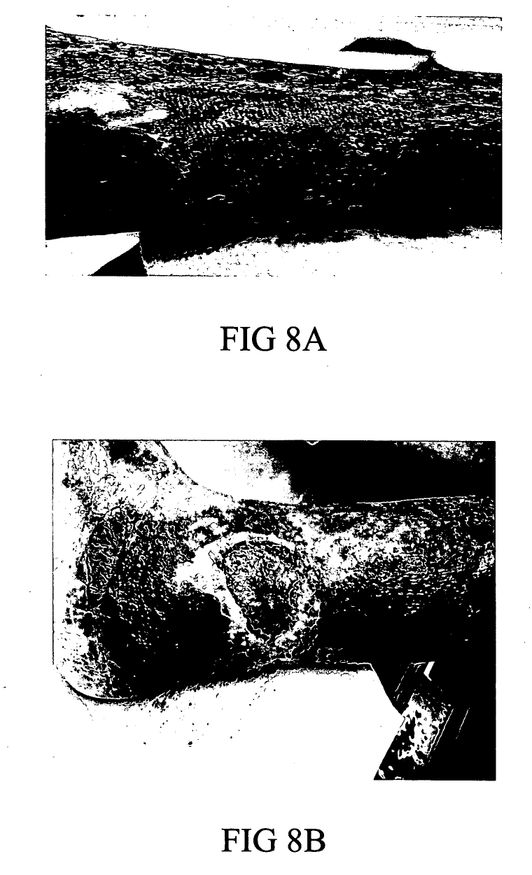

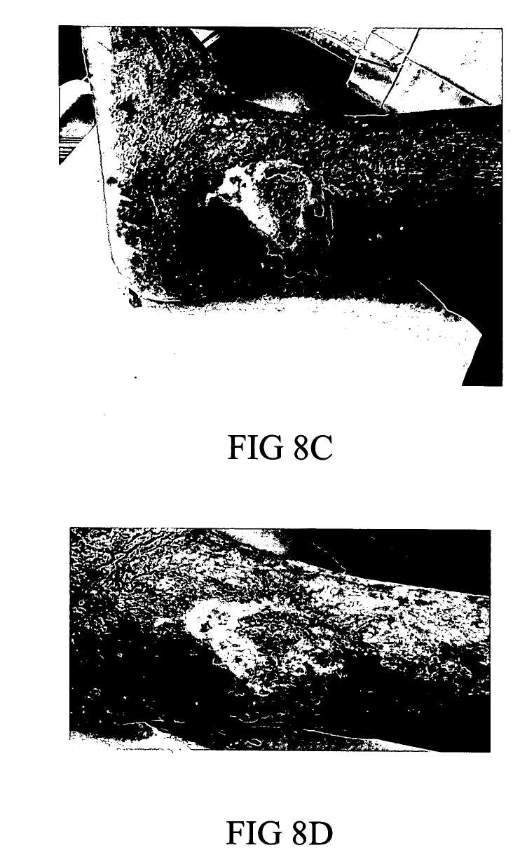

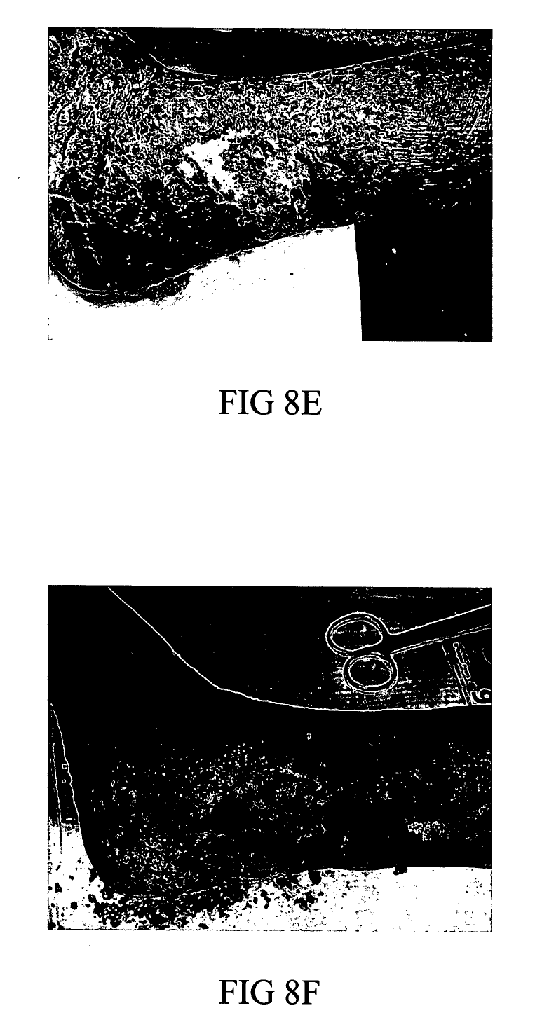

The same wound dressing A described in Example 6 was applied on a severe venous ulcer. The patient has had the ulcer for at least 4 years. The patient has 4 underlying etiologies, (Diabetes, Venous insufficiency, Livedoid vasculitis, and Arterial disease with focal stenosis). Numerous advance dressings had been applied on the ulcer without success. Dressing A (same Dressing A described in Example 6) was applied on the venous ulcer and was found to be excellent for its absorbency and anti-bacterial properties (the protocol used was to debride the wound bed, clean and apply Dressing A. This protocol is performed two times a week). No antibiotic was required. The granulation tissue that resulted was outstanding, very vascular and clean in appearance and not hypergranular. As seen from the photographs of FIG. 8, the wound showed steady healing.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

U.S. Pat. No. 2,491,416
U.S. Pat. No. 6,187,347
U.S. Pat. No. 5,763,411
U.S. Pat. No. 5,804,428
U.S. Pat. No. 5,962,026
U.S. Pat. No. 5,692,302
U.S. Pat. No. 5,874,479
U.S. Pat. No. 5,981,606
U.S. Pat. No. 5,484,913
U.S. Pat. No. 5,474,782
U.S. Pat. No. 2,163,588
U.S. Pat. No. 2,688,586
U.S. Pat. No. 2,772,999
U.S. Pat. No. 2,773,000
U.S. Pat. No. 3,206,361
U.S. Pat. No. 3,328,259
U.S. Pat. No. 4,265,233
U.S. Pat. No. 4,655,211
U.S. Pat. No. 4,463,320
U.S. Pat. No. 5,679,372
U.S. Pat. No. 5,800,372
U.S. Pat. No. 4,291,980
U.S. Pat. No. 2,366,007
U.S. Pat. No. 3,463,320
U.S. Pat. No. 4,545,974
U.S. Pat. No. 6,521,265
Remington's Pharmaceutical Sciences (17th Ed. 1985), p. 1580
U.S. Pharmacopeia Test Number 51—Antimicrobial Effectiveness Testing; pp. 1809 to 1811, USP 24 NF19 U.S. Pharmacopeia & National Formulary—Year 2000, Published by U.S. Pharmacopeial Convention Inc.

We claim:

1. A composition comprising a salt ferrate and a cation exchange material.

2. The composition according to claim 1, wherein said salt ferrate has the formula selected from the group consisting of M₂FeO₄, MFeO₄, M₂(FeO₄)₃, wherein M is a cation.

4. The composition according to claim 2, wherein said cation is selected from the group consisting of H, Li, Na, K, Ru, Cs, Fr, Be, Sr, Ti, Mn, Ni, Ga, Nb, Ru, Ag, Sn, W, Ir, Hg, Bi, NH₄, Mg, Ba, V, Fe, Cu, Ge, Mo, Rh, Cd, Hf, Re, Pt, Tl, Al, N(C₄H₉)₄, Ca, Ra, Cr, Co, Zn, Zr, Tc, Pd, In, Ta, Os, Au, Pb, and As.

5. The composition according to claim 2, wherein said cation exchange material comprises a water insoluble polymer containing one or more anionic functional groups.

6. The composition according to claim 5, wherein said anionic functional group is selected from the group consisting of SO₃⁻, OPO₃⁻, and COO⁻.

7. The composition according to claim 5, wherein said water insoluble polymers are cross-linked.

8. The composition according to claim 1, wherein said cation exchange material is an ion exchange resin in the hydrogen ionic form of a sulfonated styrene divinylbenzene copolymer.

9. The composition according to claim 1, wherein said composition is provided in substantially anhydrous condition.

10. The composition according to claim 1, wherein said composition further comprises a compound or agent that provides for antimicrobial, absorptive, and/or wound healing properties.

11. The composition according to claim 1, wherein said composition further comprises a silver compound.

12. The composition according to claim 11, wherein said silver compound is selected from the group consisting of silver metal, silver chloride, silver oxide, silver sodium hydrogen zirconium phosphate, a zirconium phosphate-based ceramic ion-exchange resin containing silver, and silver/zinc form of Zeolite A.

13. The composition according to claim 12, wherein said silver/zinc form of Zeolite A comprises silver and zinc ions encapsulated in a cross-linked polymer.

14. The composition according to claim 13, wherein said cross-linked polymer comprises vinyl pyrrolidine and methyl methacrylate.

15. The composition according to claim 13, wherein said silver/zinc form of Zeolite A comprises about 2.5% by weight of silver ion and about 14% by weight of zinc ion.

16. The composition according to claim 1, wherein said composition comprises between about 2% to about 30% by weight potassium ferrate, about 40% to about 97% by weight H⁺ polystyrene-sulfonic acid cationic exchange resin, and about 1% to about 30% by weight coated silver Zeolite A

17. The composition according to claim 1, wherein said composition further comprises one or more compound selected from the group consisting of zinc compounds, manganese compounds, calcium compounds, and copper compounds, or derivatives thereof, alginates, carrageenans, agars, chitosan, absorption polymers, natural and/or synthetic gums, oxidized celluloses, starches, and drugs or pharmaceutical compounds.

18. A method for treating a wound, said method comprising contacting said wound with an effective amount of a composition according to claim 1.

19. The method according to claim 18, wherein said wound is a pressure ulcer, venous ulcer, diabetic ulcer, ischaemic leg ulcers, ulcerative keratitis, dermal lesion, trauma or donor site injury.

20. The method according to claim 18, wherein said wound is an exuding wound, a dry wound, or a chronic wound.

21. The method according to claim 18, wherein said composition is provided in substantially anhydrous form.

22. The method according to claim 18, wherein said wound is first wetted with a suitable liquid prior to contacting said wound with said composition.

23. The method according to claim 22, wherein said suitable liquid is a saline solution or sterile water.

24. The method according to claim 18, wherein said wound is covered with a bandage or other suitable wound covering following contacting of said wound with said composition.

25. The method according to claim 18, wherein said wound is a burn or surgical incision.

26. The method according to claim 18, wherein said wound is a bite or sting from an animal, such as a dog, cat, fish, shark, snake, bat, insect, spider, scorpion, or a jellyfish; or from a plant that contains skin irritants and other toxic chemicals.

27. The method according to claim 18, wherein said composition is provided in the form of a wound covering or wound dressing impregnated with or coated with a dry powder form of a composition according to claim 1.

28. A method for absorbing exudate from an exuding wound, or for promoting granulation and epithelialization of a wound, or for accelerating healing of a wound, said method comprising contacting said wound with an effective amount of a composition according to claim 1.

29. The method according to claim 28, wherein said wound is a stage 2 or stage 3 pressure ulcer.

30. The method according to 28, wherein said wound is a second degree or third degree burn.

31. A kit comprising in one or more containers or packages a composition according to claim 1, and optionally, a suitable liquid or aqueous media for application to a dry or non-exuding wound, and optionally, a wound covering, dressing, or other wound protective material.

32. The kit according to claim 31, wherein said composition is provided in said container or package in a substantially anhydrous form.

33. A wound covering or dressing comprising a wound covering or dressing impregnated or coated with a composition according to claim 1.
